(19) United States
(12) Patent Application Publication
(10) Pub. No.: US 2026/0035332 A1
(43) Pub. Date: Feb. 5, 2026

(54) ZONE CATALYST LOADING AND PROCESSES FOR THE ACETOXYLATION OF OLEFINS USING THE SAME

(71) Applicant: Celanese International Corporation, Irving, TX (US)

(72) Inventors: Steve R. Alexander, Irving, TX (US); Laiyuan Chen, Irving, TX (US); Stacey Somerville, Irving, TX (US); Sean Mueller, Irving, TX (US)

(73) Assignee: Celanese International Corporation, Irving, TX (US)

(21) Appl. No.: 19/100,282

(22) PCT Filed: Aug. 1, 2023
(86) PCT No.: PCT/US2023/029227

§ 371 Date: Jan. 31, 2025

Related U.S. Application Data
(60) Provisional application No. 63/395,485, filed on Aug. 5, 2022.

(51) Int. Cl. C07C 67/055; B01J 23/44; B01J 23/52

(52) U.S. Cl. CPC C07C 67/055; B01J 23/44; B01J 23/52

(57) ABSTRACT

Disclosed herein is a process for the acetoxylation of olefins in a gaseous reaction stream containing an olefin, acetic acid, and an oxygen-containing gas. The process comprises passing a reaction gas over at least two fixed catalyst zones, arranged in series. The catalyst zones are located in one or more reaction tubes arranged in parallel. The at least two fixed catalyst zones comprise an inlet catalyst zone comprising an inlet catalyst and an outlet catalyst zone comprising an outlet catalyst and certain conditions may be met for the inlet and outlet catalyst.

### PRIORITY CLAIM

This Application claims priority to U.S. Provisional Application No. 63/395,485, filed on Aug. 5, 2022, the entire contents and disclosures of which are incorporated by reference herein.

### FIELD OF THE INVENTION

The invention relates to zone catalyst loading and to processes of acetoxylating olefins using the same. In particular, the invention relates to zone catalyst loading with at least two fixed catalyst zones, wherein at least one of the following conditions is met:(a) the inlet catalyst comprises greater than 7 g/L:Pd; (b) the outlet catalyst comprises greater than 8.3 g/L Pd; and/or (c) the outlet catalyst comprises from 7.5 to 8.1 g/L Pd.

### BACKGROUND OF THE INVENTION

Processes for the acetoxylation of ethylene in the gas phase are of particular industrial interest. Acetoxylations may be carried out industrially by catalytic gas-phase oxidation of olefins, such as ethylene or propylene, in fixed-bed reactors. These reactions may be carried out in shell-and-tube reactors. Unsaturated esters, such as vinyl acetate, may be prepared by this reaction. In general, the reaction is carried out by passing a gaseous mixture comprising molecular oxygen, an olefin and acetic acid through a reactor. The reaction is typically conducted in a shell-and-tube reactor in which a plurality of reaction tubes are arranged in parallel and in each of which a uniform catalyst charge is located. The excess heat of reaction involved is removed by means of a heat transfer medium. An example of such a reactor is a boiling water reactor.

Despite various methods of regulating the reaction temperature, differences in temperature may still occur during the reaction. These temperature peaks (known as “hot spots”) may bring about a series of undesirable effects during the course of the reaction. Hot spots may prematurely age the catalyst, reduce selectivity and productivity, and limit space-time yield. Processes for the acetoxylation of olefins, particularly of ethylene, in the gas phase which lead high yields of vinyl acetate are of great economic importance.

WO 2008/071610 discloses a process and a catalyst system comprising a catalyst which comprises palladium, gold and potassium acetate and is applied to an SiO₂ support having a large surface area and may be operated at a space-time yield of more than 800 [g (VAM)/l cat*h] at ethylene selectivities of greater than 92% and at a low degree of formation of ethyl acetate relative to vinyl acetate.

U.S. Pat. No. 5,179,056 discloses a process for preparing vinyl acetate by reaction of ethylene and acetic acid in the presence of an oxygen-containing gas over a highly reactive palladium/gold coated catalyst.

U.S. Pat. No. 6,399,813 discloses a highly active fluidized-bed vinyl acetate catalyst on a support composed of inert microspheroidal particles composed of silicon oxide, zirconium oxide or aluminum oxide and having a defined pore distribution.

In view of the great economic importance of acetoxylated products and the high-performance catalysts known from the prior art, there is a great need to optimize the course of the reaction in respect of conversion, selectivity and life of the catalyst.

WO 2007/101749 and WO 2006/042659 disclose, for example, synthesis reactors for preparing vinyl acetate monomer with increased selectivity and productivity, in which gaseous ethylene and acetic acid and also an oxygen-containing gas react catalytically, with the synthesis reactors being a wall reactor and the catalytic synthesis being carried out in a plurality of reaction spaces and at least one wall of the reaction spaces being coated with catalyst and at least one wall of the reaction spaces being indirectly cooled.

U.S. Pat. No. 8,907,123 discloses a process for the acetoxylation of olefins in a gaseous reaction stream containing an olefin, acetic acid and an oxygen-containing gas, comprising passing a reaction gas comprising at least one olefin, oxygen, and acetic acid over at least two fixed catalyst zones of supported olefin acetoxylation catalysts of differing activity arranged in series, wherein the catalyst zones are located in or more reaction tubes arranged in parallel.

The need remains for catalyst zone loading that leads to desirable selectivity, productivity, and space-time yield for the acetoxylation of olefins. These and other issues are disclosed in the present specification.

### BRIEF SUMMARY OF THE INVENTION

In some embodiments, the present disclosure includes a process for the acetoxylation of olefins in a gaseous reaction stream containing an olefin, acetic acid, and an oxygen-containing gas, the process comprising: passing a reaction gas over at least two fixed catalyst zones, arranged in series, wherein the catalyst zones are located in one or more reaction tubes arranged in parallel; wherein the at least two fixed catalyst zones comprise an inlet catalyst zone comprising an inlet catalyst and an outlet catalyst zone comprising an outlet catalyst; wherein the inlet catalyst zone comprises from 5 to 70% of the catalyst loading and the outlet catalyst zone comprises from 30 to 95% of the catalyst loading, based on 100% by weight of catalyst loading of all catalyst zones; and wherein at least one of the following conditions is met:(a) the inlet catalyst comprises greater than 7 g/L; Pd; (b) the outlet catalyst comprises greater than 8.3 g/L Pd; and/or (c) the outlet catalyst comprises from 7.5 to 8.1 g/L Pd. The inlet catalyst zone may comprise from 40 to 70% by weight of the catalyst loading. In some aspects, condition (b) and/or (c) is met and the inlet catalyst comprises from 4 to 15 g/L Pd and from 2 to 8 g/L Au. In some aspects, the outlet catalyst zone comprises from 30 to 50% by weight of the catalyst loading. In some aspects, condition (a) is met and the outlet catalyst comprises from 4 to 15 g/L Pd and from 2 to 8 g/L Au. In some aspects, the inlet catalyst zone comprises from 5 to 40% by weight of the catalyst loading. In some aspects, the inlet catalyst has a weight ratio of Au to Pd from 0.3:1 to 1.5:1. In some aspects, the inlet catalyst has a layer thickness from 100 to 300 micrometers. In some aspects, the inlet catalyst zone contains at least two catalysts in series and the weight ratio of the two catalysts is from 1:5 to 5:1. In some aspects, the outlet catalyst zone comprises from 60 to 95% by weight of the catalyst loading. In some aspects, the outlet catalyst has a weight ratio of Au to Pd of 0.3:1 to 1.5:1. In some aspects, the inlet catalyst has a different metal loading than the outlet catalyst. In some aspects, the inlet catalyst zone comprises a catalyst having a different size than the outlet catalyst zone. In some aspects, the inlet catalyst and outlet catalyst each comprise less than 2 wt. % Pd. In some aspects, the inlet catalyst and outlet catalyst each comprise from 25 to 55 g/L potassium acetate. In some aspects, the olefin is ethylene and the selectivity to vinyl acetate monomer is at least 60%. In some aspects, the olefin is ethylene and the conversion of ethylene is at least 60%. In some aspects, the olefin is ethylene and the selectivity to heavy ends is less than 10%. In some aspects, the inlet catalyst and the outlet catalyst have a spherical shape. In some aspects, the olefin is ethylene and the oxygen conversion is from 35 to 55%. In some aspects, only condition a) is met. In some aspects, only condition b) is met. In some aspects, only condition c) is met. In some aspects, conditions a) and b) are met. In some aspects, conditions a) and c) are met.

Additional embodiments and features are set forth in part in the description that follows, and in part will become apparent to those skilled in the art upon examination of the specification or may be learned by the practice of the invention. The features and advantages of the invention may be realized and attained by means of the instrumentalities, combinations, and methods described in the specification.

### BRIEF DESCRIPTION OF THE DRAWINGS

A further understanding of the nature and advantages of the present invention may be realized by reference to the remaining portions of the specification and the drawings wherein like reference numerals are used throughout the several drawings to refer to similar components. In some instances, a sublabel is associated with a reference numeral and follows a hyphen to denote one of multiple similar components. When reference is made to a reference numeral without specification to an existing sublabel, it is intended to refer to all such multiple similar components.

FIG. 1 is a schematic of the process used to form vinyl acetate monomer in accordance with embodiments of the present disclosure.

FIG. 2 is an illustration of two catalyst zones in accordance with embodiments of the present disclosure.

FIG. 3 is an illustration of three catalyst zones in accordance with embodiments of the present disclosure.

FIG. 4 is an illustration of a plurality of parallel reaction tubes in accordance with embodiments of the present disclosure.

### DETAILED DESCRIPTION OF THE INVENTION

### Introduction

As described herein, the present disclosure relates to zone catalyst loading and to processes for acetoxylating olefins using zone catalyst loading. The process may comprise acetoxylating olefins in a gaseous stream. The gaseous stream may contain an olefin, acetic acid, and an oxygen-containing gas. The process steps may include: 1) passing the gaseous reaction stream over at least two fixed catalyst zones. The at least two fixed catalyst zones are arranged in series. The catalyst zones are located in one or more reaction tubes. The tubes may be arranged in parallel. The at least two fixed catalyst zones may comprise an inlet catalyst zone comprising an inlet catalyst and an outlet catalyst zone comprising an outlet catalyst. The inlet catalyst zone may comprise from 5 to 70% by weight of the catalyst loading, based on 100% by weight of catalyst loading in all catalyst zones. To form the product, at least one of the following conditions may be met in the:(a) the inlet catalyst comprises greater than 7 g/L:Pd; (b) the outlet catalyst comprises greater than 8.3 g/L Pd; and/or (c) the outlet catalyst comprises from 7.5 to 8.1 g/L Pd.

Without being bound by theory, it is believed that by using two or more catalyst zones as described herein, the catalyst workload may be flattened along the length of the reaction tube. A flatter temperature profile may lead to better control of the heat moving through the reaction tube and may improve heat removal capacity. By using two or more catalyst zones as described herein, the catalyst aging pattern may be made more even, thus leading to greater selectivity and/or productivity over time. The catalysts chosen for the catalyst zones may be different catalysts, in terms of metal content, or may be sized, shaped, or prepared differently. In some aspects, the catalyst is selected in order to balance catalyst life, product yield, and metal loss during fabrication. These and other details are described further herein.

### Acetoxylation of Olefins

As described herein, the zone catalyst loading may be used in a process for the acetoxylation of olefins. Exemplary olefins include ethylene and propylene, though others are also contemplated. The acetoxylation of ethylene to form vinyl acetate monomer is of particular industrial interest. The vinyl acetate monomer (VAM) is a compound represented by the following formula:

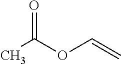

VAM is an important component in a wide variety of products, including polymers. VAM is also an important intermediate in coatings, textiles, paints, and other applications. For example, the polymer of VAM, polyvinyl acetate, is used in myriad applications including glues and adhesives.

The reaction to form VAM includes forming a gaseous reaction stream, e.g., a reaction gas. The reaction gas may comprise ethylene, acetic acid, and an oxygen-containing gas, e.g., molecular oxygen. In some aspects, the reaction gas may further comprise inert gases. The reaction gas may then be passed over at least two fixed catalyst zones, wherein the at least two fixed catalyst zones are arranged in series. The at least two fixed catalyst zones are located in a reaction tube. There may be multiple reaction tubes, arranged in parallel. Each reaction tube may comprise the same at least two fixed catalyst zones. The at least two fixed catalyst zones may comprise an inlet catalyst zone and an outlet catalyst zone. In the cases where more than two catalyst zones are present, the additional catalyst zones are understood to be between the inlet catalyst zone and outlet catalyst zone. Each catalyst zone comprises at least one catalyst.

The amount of catalyst loading in each catalyst zone may be selected depending on desired catalyst performance. In some aspects, the inlet catalyst zone may comprise from 5 to 70% of the catalyst loading of the entire reaction tube, e.g., of all catalyst in all catalyst zones in the reaction tube. In some aspects, the outlet catalyst zone may comprise from 30 to 95% of the catalyst loading of the entire reaction tube. In some aspects, a majority of the reaction tubes comprise approximately the same catalyst zones, allowing for slight differences based on aging the catalyst in each zone.

The reaction may be conducted, in the gas phase, at a temperature from 100 to 250° C. and at a pressure from 1 to 25 bars.

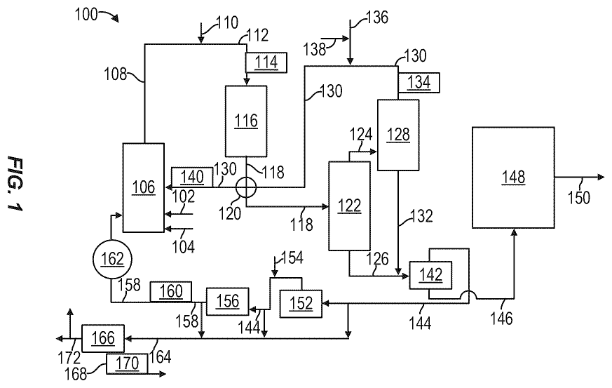

The acetoxylation of ethylene yields a crude vinyl acetate product comprising VAM water, and carbon dioxide as well as unreacted ethylene and acetic acid, which are used in excess. The ethylene and acetic acid are recycled back to the reactor from the reaction and purification sections of the unit. Product VAM is recovered and purified in the purification section and sent to storage tanks. Wastewater is sent to a treatment facility and carbon dioxide is vented to a pollution control device. Inert gases such as nitrogen and argon may accumulate over time and may then be purged from the reaction section to minimize buildup. A full description of the VAM production process is provided herein and illustrated in FIG. 1.

### VAM Production Process

FIG. 1 illustrates a process flow diagram of an example vinyl acetate production process 100 of the present disclosure. Additional components and modifications may be made to the process 100 without changing the scope of the present invention. Further, as would be recognized by one skilled in the art, the description of the process 100 and related system uses streams to describe the fluids passing through various lines. For each stream, the related system has corresponding lines (e.g., pipes or other pathways through which the corresponding fluids or other materials may pass readily) and optionally valves, pumps, compressors, heat exchangers, or other equipment to ensure proper operation of the system whether explicitly described or not.

Further, the descriptor used for individual streams does not limit the composition of said streams to consisting of said descriptor. For example, an ethylene stream does not necessarily consist of only ethylene. Rather, the ethylene stream may comprise ethylene and a diluent gas (e.g., an inert gas). Alternatively, the ethylene stream may consist of only ethylene. Alternatively, the ethylene stream may comprise ethylene, another reactant, and optionally an inert component.

In the illustrated process 100, an acetic acid stream 102 and an ethylene stream 104 are introduced to a vaporizer 106. Optionally, ethane may also be added to the vaporizer 106. In addition, one or more recycle streams 130, 158 (each described further herein) may also be introduced to the vaporizer 106. Optionally, one or more of the recycle streams 130, 158 may be combined with the acetic acid stream 102 (not shown) before introduction to the vaporizer 106.

The temperature and pressure of vaporizer 106 may vary over a wide range. The vaporizer 106 preferably operates at a temperature from 100° C. to 250° C., or from 100° C. to 200° C., or from 120° C. to 150° C. The operating pressure of the vaporizer 106 may range from 0.1 MPa to 2.03 MPa, or 0.25 MPa to 1.75 MPa, or 0.5 MPa to 1.5 MPa. The vaporizer 106 produces a vaporized feed stream 108. The vaporized feed stream 108 exits the vaporizer 106 and combines with an oxygen stream 110 to produce a combined feed stream 112. The combined feed stream 112 is analyzed by sensors 114 prior to being fed to a vinyl acetate reactor 116.

The sensors 114 include a water sensor for determining the concentration of water in the combined feed stream 112. The sensors 114 may optionally also include a temperature sensor, a pressure sensor, a flow rate sensor, composition sensors (e.g., gas chromatography, infrared spectroscopy, and oxygen analyzers) and the like, and any combination thereof. Each of the individual sensors may be present singularly or in a plurality. Having a plurality of a specific sensor provides redundancies that minimize downtime for sensor replacement and mitigate safety issues resulting from a failing or out of calibration sensor. While the sensors 114 are generally illustrated as upstream of the vinyl acetate reactor 116, said sensors may be placed in other locations where calculations may be performed to estimate a condition (e.g., temperature, pressure, or component concentration) at the reactor inlet or other suitable location.

The operating conditions in the vinyl acetate reactor 116 may be adjusted based on the composition of the combined feed stream 112. Generally, suitable ranges for the operating conditions in the vinyl acetate reactor 116 are provided below.

Regarding the general operating conditions of the vinyl acetate reactor 116, the molar ratio of ethylene to oxygen when producing vinyl acetate is preferably less than 20:1 in the vinyl acetate reactor 116 (e.g., 1:1 to 20:1, or 1:1 to 10:1, or 1.5:1 to 5:1, or 2:1 to 4:1). Further, the molar ratio of acetic acid to oxygen is preferably less than 10:1 in the vinyl acetate reactor 116 (e.g., 0.5:1 to 10:1, 0.5:1 to 5:1, or 0.5:1 to 3:1). The molar ratio of ethylene to acetic acid is preferably less than 10:1 in the vinyl acetate reactor 116 (e.g., 1:1 to 10:1, or 1:1 to 5:1, or 2:1 to 3:1). Accordingly, the combined feed stream 112 comprise the ethylene, oxygen, and acetic acid in said molar ratios.

The vinyl acetate reactor 116 may be a shell and tube reactor that is capable, through a heat exchange medium, of absorbing heat generated by the exothermic reaction and controlling the temperature therein within a temperature range of 100° C. to 250° C., or 110° C. to 200° C., or 120° C. to 180° C. The pressure in the vinyl acetate reactor 116 may be maintained at 0.5 MPa to 2.5 MPa, or 0.5 MPa to 2 MPa.

Further, the vinyl acetate reactor 116 may be a fixed bed reactor or a fluidized bed reactor, preferably a fixed bed reactor that containing reaction tubes as described herein.

Reactor 116 may comprise one or more tubes, e.g., a plurality of tubes arranged in parallel. In some aspects, the reactor may comprise from 2 to 10,000 tubes, e.g., from 50 to 10,000 tubes, from 500 to 10,000 tubes, from 1000 to 9500 tubes, or from 3000 to 9000 tubes.

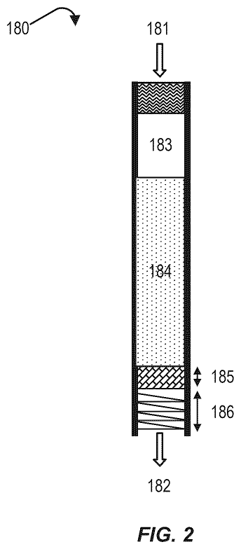

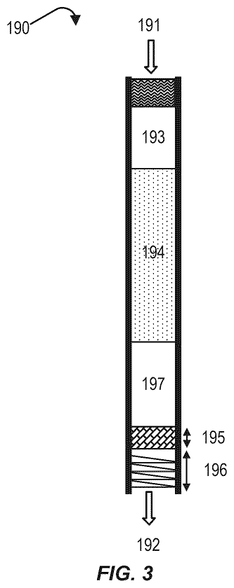

Each tube comprises the at least two fixed catalyst zones, described herein. In some aspects, at least 10% of the tubes comprise the same at least two fixed catalyst zones, e.g., at least 20%, at least 30%, at least 40%, at least 50%, at least 60%, at least 70%, at least 80%, at least 90%, at least 95%, at least 97.5%, at least 99%, at least 99.5%, or 100%, based on the total number of catalyst tubes. Two exemplary but non-limiting catalyst tube configurations as shown in FIG. 2 (tube 180) and FIG. 3 (tube 190). FIG. 2 shows a tube 180 showing inlet flow 181 into an open portion of the tube which may comprise inerts. These inerts may serve to filter the incoming flow to remove solids or liquids, similarly to a guard bed. Inlet catalyst zone 183 contains a fixed inlet catalyst. Outlet catalyst zone 184 contains a fixed outlet catalyst. The reaction gas then passes inerts 185, which may support the catalyst to prevent it from moving into/through the spring 186. The reaction gas then exits tube 180 in the direction of outlet flow 182. Similarly, FIG. 3 shows another non-limiting zone loaded catalyst configuration with tube 190 having an inlet flow 191, an inlet catalyst zone 193 comprising an inlet catalyst, an additional catalyst zone 194 comprising an additional catalyst, an outlet catalyst zone 197 comprising an outlet catalyst, inerts 195, spring 196, and outlet flow 192.

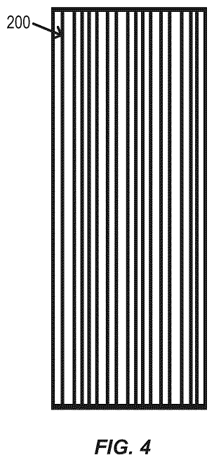

A multiple tube arrangement is shown in FIG. 4. FIG. 4 shows a plurality of tubes 200 in parallel but is not intended to be limiting in terms of the number of tubes. Tube 200 may be the tube 180, tube 190, or another tube configuration described herein.

Referring again to FIG. 1, because the process 100 uses one or more recycle streams 130, 156, 168 and diluents may be included, the components in the combined feed stream 112 are more than ethylene, acetic acid, and oxygen. Examples of components in the combined feed stream 112 where the concentration of said components may include, but are not limited to, ethylene, acetic acid, methane, ethane, propane, water, nitrogen, argon, and carbon dioxide.

Referring again to FIG. 1, the vinyl acetate reaction in the reactor 116 produces a crude vinyl acetate stream 118. Depending on conversion and reaction conditions, the crude vinyl acetate stream 118 may comprise 5 wt. % to 30 wt. % vinyl acetate, 5 wt. % to 40 wt. % acetic acid, 0.1 wt. % to 10 wt. % water, 10 wt. % to 80 wt. % ethylene, 1 wt. % to 40 wt. % carbon dioxide, 0.1 wt. % to 50 wt. % alkanes (e.g., methane, ethane, or mixtures thereof), and 0.1 wt. % to 15 wt. % oxygen. Optionally, the crude vinyl acetate stream 118 may also comprise 0.01 wt. % to 10 wt. % ethyl acetate. The crude vinyl acetate stream 118 may comprise other compounds such as methyl acetate, acetaldehyde, acrolein, propane, and inerts such as nitrogen or argon. Generally, these other compounds, except for inerts, are present in very low amounts.

The crude vinyl acetate stream 118 passes through a heat exchanger 120 to reduce the temperature of the crude vinyl acetate stream 118 and then to a separator 122 (e.g., a distillation column). Preferably, the crude vinyl acetate stream 118 is cooled to a temperature of 80° C. to 145° C., or 90° C. to 135° C., prior to being introduced into the separator 122. Preferably, no condensation of the liquefiable components occurs and the cooled crude vinyl acetate stream 118 is introduced to the separator 122 as gas.

The energy to separate the components of the crude vinyl acetate stream 118 may be provided by the heat of reaction in the reactor 116. In some embodiments, there may be an optional reboiler dedicated to increasing the separation energy within the separator 122. [0049] The separator 122 separates the crude vinyl acetate stream 118 into at least two streams: an overheads stream 124 and a bottoms stream 126. The overheads stream 124 may comprise ethylene, carbon dioxide, water, alkanes (e.g., methane, ethane, propane or mixtures thereof), oxygen, and vinyl acetate. The bottoms stream may comprise vinyl acetate, acetic acid, water, and potentially ethylene, carbon dioxide, and alkanes.

The overheads stream 124 is conveyed to a scrubber 128 to remove vinyl acetate in the overheads stream 124. As a result, the scrubber 128 has gas stream 130 and a bottoms stream 132. Vinyl acetate scrubbing may be achieved by passing the overheads stream 124 through a mixture of water and acetic acid.

The tail gas stream 130 comprises ethylene, carbon dioxide, alkanes, and oxygen. The conditions (e.g., temperature, pressure, and/or composition of components) of the tail gas stream 130 may be measured using sensors 134. The sensors 134 may include, but are not limited to, a temperature sensor, a pressure sensor, a flow rate sensor, composition sensors (e.g., gas chromatography, infrared spectroscopy, and oxygen analyzers) and the like, and any combination thereof. Each of the individual sensors may be present singularly or in a plurality. Having a plurality of a specific sensor provides redundancies that minimize downtime for sensor replacement and mitigate safety issues resulting from a failing or out of calibration sensor. While the sensors 134 are generally illustrated as downstream of the scrubber 128 along the tail gas stream 130, said sensors may be placed in other locations where calculations may be performed to estimate a condition (e.g., temperature, pressure, or component concentration) of the tail gas stream 130 post scrubber 128.

The tail gas stream 130 (also referred to as a recycle stream) is conveyed back to the vaporizer 106 through the heat exchanger 120, where the crude vinyl acetate stream 118 heats the tail gas stream 130. Optionally, the tail gas stream 130 may be augmented with or otherwise have added thereto other streams including other recycle streams (not shown) in the process and feed streams. As illustrated, an ethylene feed steam 136 and a methane feed stream 138 (or other ballast gas stream) are combined (e.g., mixed with or entrained with) with the tail gas stream 130.

Additionally, between the scrubber 128 and the heat exchanger 120, other processes (not illustrated) may be performed on the tail gas stream 130. For example, the tail gas stream 130 may have at least a portion of the carbon dioxide removed. [0054]

Between the heat exchanger 120 and the vaporizer 106, the tail gas stream 130 is analyzed by sensors 140. Examples of sensors 140 include, but are not limited to, a temperature sensor, a pressure sensor, a flow rate sensor, composition sensors (e.g., gas chromatography, infrared spectroscopy, and oxygen analyzers) and the like, and any combination thereof. Each of the individual sensors may be present singularly or in a plurality. Having a plurality of a specific sensor provides redundancies that minimize downtime for sensor replacement and mitigate safety issues resulting from a failing or out of calibration sensor. While the sensors 140 are generally illustrated as between the heat exchanger 120 and the vaporizer 106, said sensors may be placed in other locations where calculations may be performed to estimate a condition (e.g., temperature, pressure, or component concentration) between the heat exchanger 120 and the vaporizer 106 or other suitable location.

Referring again to FIG. 1, the bottoms stream 126 from the separator 122 and the bottoms stream 132 from the scrubber 128 may be combined and fed to a crude tank 142.

Generally, the stream(s) coming into the crude tank 142 are depressurized to a pressure of 0.1 MPa to 0.15 MPa. In depressurizing the incoming stream, the ethylene, carbon dioxide, inert gases (e.g., nitrogen and/or argon), and acetic acid flash to produce a flash gas stream 144. The bottoms of the crude tank 142 primarily comprise vinyl acetate, water, and acetic acid with some ethyl acetate byproduct. The bottoms are transported as a vinyl acetate stream 146 to be purified by various processes 148 to produce the purified vinyl acetate product stream 150. Examples of purification processes 148 include, but are not limited to, azeotrope distillation, water stripping, distillation, phase separations, and the like, and any combination thereof. Examples of different processing methods and systems are described in U.S. Pat. Nos. 6,410,817, 8,993,796, and 9,045,413 and US Patent App. Pub. No. 2014/0066649, each of which is incorporated herein by reference.

Further, the purification processes 148 may produce additional streams that individually or in any combination may be recycled back to the vaporizer 106, the tail gas stream 130, the flash gas stream 144, and/or other streams within the process 100.

Optionally (not shown), a portion of the tail gas slip stream 130 may be combined with (e.g. mixed with or entrained with) the flash gas stream 144.

At least a portion of the carbon dioxide in the flash gas stream 144 (optionally having been combined with a portion of the tail gas slip stream 130) is removed before recycling back into the vaporizer 106. As illustrated, the flash gas stream 144 first passes through a CO₂ scrubber 152 and then a CO₂ absorber 156 to produce a CO₂ removal overheads stream 158. Between the CO₂ scrubber 152 and the CO₂ absorber 156, ethylene may be added to the flash gas stream 144 from ethylene stream 154.

After the CO₂ absorber 156, the CO₂ removal overheads stream 158 is analyzed by sensors 160. Examples of sensors 160 include, but are not limited to, a temperature sensor, a pressure sensor, a flow rate sensor, composition sensors (e.g., gas chromatography, infrared spectroscopy, and oxygen analyzers) and the like, and any combination thereof. Each of the individual sensors may be present singularly or in a plurality. Having a plurality of a specific sensor provides redundancies that minimize downtime for sensor replacement and mitigate safety issues resulting from a failing or out of calibration sensor. While the sensors 160 are generally illustrated as downstream of the CO₂ absorber 156, said sensors 160 may be placed in other locations where calculations may be performed to estimate a condition (e.g., temperature, pressure, or component concentration) downstream of the CO₂ absorber 156 or other suitable location.

Referring again to FIG. 1, the CO₂ removal overheads stream 158 may then be passed through a heat exchanger 162 and fed into the vaporizer 106. Further, a slip stream 164 from the flash gas stream 144 and/or the CO₂ removal overheads stream 158 is used to purge inerts from the system. This slip stream 164 may be sent through an ethylene recovery process 166. The ethylene recovery process 162 produces an ethylene vent stream 168 and a recycle stream 172. Examples of ethylene recovery processes 166 may include, but are not limited to, scrubbing systems, membrane recovery processes, and the like, and any combination thereof. The ethylene recovery processes 166 may produce a vent stream 168 and additional stream(s) 172 that take the ethylene recovered to other processes or for recycling back into this process 100.

Accordingly, after the ethylene recovery process 166, the ethylene vent stream 168 is analyzed by sensors 170. Examples of sensors 170 include, but are not limited to, a temperature sensor, a pressure sensor, a flow rate sensor, composition sensors (e.g., gas chromatography, infrared spectroscopy, and oxygen analyzers) and the like, and any combination thereof. Each of the individual sensors may be present singularly or in a plurality. Having a plurality of a specific sensor provides redundancies that minimize downtime for sensor replacement and mitigate safety issues resulting from a failing or out of calibration sensor. While the sensors 170 are generally illustrated along the ethylene vent stream 168, said sensors 170 may be placed in other locations where calculations may be performed to estimate a condition (e.g., temperature, pressure, or component concentration) ethylene vent stream 168 or other suitable location.

### Zone Loaded Catalyst(s)

As illustrated in FIG. 2, tube 180 comprises an inlet catalyst zone 183 and an outlet catalyze zone 184. The inlet catalyst zone may comprise one or more catalysts, referred to as inlet catalysts. Similarly, the outlet catalyst zone may comprise one or more catalysts, referred to as outlet catalysts. It is also contemplated, as shown in FIG. 3, that one or more catalyst zones may be included between the inlet catalyst zone and outlet catalyst zone, referred to as additional catalyst zone(s), e.g., first additional catalyst zone, second additional catalyst zone, etc. Each catalyst zone may contain a single catalyst.

In some aspects, the inlet catalyst zone comprises from 5 to 70% of the catalyst loading, based on 100% by weight of the catalyst loading of all catalyst zones, e.g., from 10 to 70%, from 15 to 70%, from 20 to 70%, from 25 to 70%, from 30 to 70%, from 35 to 70%, from 40 to 70%, from 45 to 70%, from 50 to 70%, from 50 to 65%, from 15 to 65%, from 15 to 60%, from 15 to 55%, from 20 to 50%, from 20 to 40%, from 20 to 35%, or from 20 to 30%.

In some aspects, the outlet catalyst zone comprises from 30 to 95% of the catalyst loading, based on 100% by weight of the catalyst loading of all catalyst zones, e.g., from 35 to 90%, from 35 to 80%, from 40 to 80%, from 40 to 75%, from 40 to 70%, from 45 to 70%, from 50 to 70%, from 55 to 70%, from 55 to 95%, from 55 to 90%, from 55 to 85%, from 55 to 80%, from 60 to 80%, from 65 to 80%, or from 70 to 80%.

In some aspects, at least one additional catalyst zone may be present. In such aspects, the at least one additional catalyst zone may comprise from 1 to 25% of the catalyst loading, based on the catalyst loading of all catalyst zones, e.g., from 5 to 20%, or from 10 to 15%.

In some aspects, the inlet catalyst zone comprises more of the catalyst loading than the outlet catalyst zone, e.g., greater than 50% by weight based on the weight of all catalyst loading in all catalyst zones. In other aspects, the inlet catalyst zone comprises less of the catalyst loading than the outlet catalyst zone, e.g., less than 50% by weight based on the weight of all catalyst loading in all catalyst zones.

In some aspects, the inlet, outlet, and/or additional catalyst comprises palladium (Pd). In some aspects, the inlet catalyst comprises from 4 to 15 g/L Pd, e.g., from 5 to 13 g/L, from 5 to 11 g/L, from 5 to 10 g/L, from 5 to 9 g/L, from 5.5 to 8.5 g/L, from 6 to 8.5 g/L, or from 6.5 to 8.5 g/L. In terms of weight percent, the catalyst may comprise less than 2 wt. % Pd, e.g., less than 1.75 wt. %, less than 1.5 wt. %, or less than 1.25 wt. %. In terms of ranges, the catalyst may comprise from 0.1 to <2 wt. % Pd, e.g., from 0.5 to 1.75 wt. %, from 0.75 to 1.5 wt. %, or from 0.8 to 1.15 wt. %.

In some aspects, the inlet, outlet, and/or additional catalyst comprises gold (Au). The inlet catalyst may comprise from 2 to 8 g/L Au, e.g., from 2.5 to 7.5 g/L, from 3 to 7 g/L, from 3 to 6 g/L, from 3 to 5 g/L, from 3.25 to 5 g/L, from 3.5 to 4.75 g/L, or from 3.5 to 4.5 g/L. In some specific aspects, in terms of weight percent, the catalyst may comprise less than 1.5 wt. % Au, e.g., less than 1.25 wt. %, less than 1 wt. %, or less than 0.75 wt. %. In terms of ranges, the catalyst may comprise from 0.1 to 1.5 wt. % Au, e.g., from 0.2 to 1 wt. %, from 0.3 to 0.75 wt. %, or from 0.4 to 0.75 wt. %.

For purposes of calculating the weight percent of components on the catalyst, a density of 630 g/L may be used.

In some aspects, the inlet, outlet, and/or additional catalyst comprises Au and Pd. The ratio of Au to Pd may range from 0.3:1 to 1.5:1, on a g/L basis of Au to Pd, e.g., from 0.4:1 to 1.3:1, from 0.45:1 to 1.2:1, from 0.45:1 to 1:1, from 0.5:1 to 0.75:1, or from 0.5:1 to 0.6:1. In some aspects, Pd is present in a greater amount than Au, in terms of g/L and wt. %.

In some aspects, the inlet, outlet, and/or additional catalyst comprises potassium acetate (KOAc). The KOAc may be present in the greatest amount, as compared to Pd and Au. The KOAc may be present from 25 to 55 g/L, e.g., from 30 to 50 g/L, from 35 to 45 g/L, from 37.5 to 42.5 g/L, or approximately 40 g/L. In terms of weight percent, the KOAc may be present from 3.0 to 10.0 wt. %, 4.0 to 9.0 wt. %, from 4.5 to 8.5 wt. %, from 5.0 to 7.5 wt. %, or from 5.5 to 7.0 wt. %.

In some aspects, the inlet, outlet, and/or additional catalyst may contain a refractory support, including a metal oxide such as silica, silica-alumina, titania, or zirconia. In some aspects, the refractory support is silica. The refractory support may then be coated with a layer of catalyst components, e.g., Pd, Au, and KOAc. The thickness of the coating on the refractory support may range from 100 to 300 microns, e.g., from 115 to 200 microns, from 120 to 175 microns, from 125 to 150 microns, from 200 to 300 microns, or from 225 to 275 microns. In some aspects, the thickness of the coating may be selected based on the size of the catalyst. The size of the catalyst refers to the catalyst diameter in the longest direction. The catalyst diameter may be measured according to ASTM UOP947-96 (1996).

In some aspects, the inlet catalyst and/or outlet comprises the composition shown in Table 1 below.

TABLE 1
Catalyst Compositions

| | Pd (g/L) | Au (g/L) | KOAc g/L | Size (mm) |
|---|---|---|---|---|
| Catalyst A | 4 to 7 | 1 to 3 | 25-55 | 5 |
| Catalyst B | >7 to <7.5 | >3 to 4.8 | 25-55 | 5 |
| Catalyst C | 7.5 to 8 | >4.8 to <6 | 25-55 | 7 |
| Catalyst D | 7 to 10 | 3 to 8 | 25-55 | 6 |
| Catalyst E | >8 to 10 | >4.8 to <6 | 25-55 | 7 |
| Catalyst F | 7.5 to 8 | 6 to 8 | 25-55 | 5 |

In some aspects, the inlet catalyst zone may comprise Catalyst B as the inlet catalyst and the outlet catalyst zone may comprise Catalyst A, B, C, D, E, or F as the outlet catalyst. In some aspects, the inlet catalyst zone may comprise Catalyst C as the inlet catalyst and the outlet catalyst zone may comprise Catalyst A, B, C, D, E, or F as the outlet catalyst. In some aspects, the inlet catalyst zone may comprise Catalyst D as the inlet catalyst and the outlet catalyst zone may comprise Catalyst A, B, C, D, E, or F as the outlet catalyst. In some aspects, the inlet catalyst zone may comprise Catalyst A, B, C, D, E or F as the inlet catalyst and the outlet catalyst zone may comprise Catalyst E as the outlet catalyst. As disclosed in Table 1 above, the Au content may range from 2 to 8 g/L. It is expressly contemplated within this disclosure that the Au content may be selected from the subranges disclosed herein. Similarly, the other selections for the catalyst, including the support, the ratio of Au/Pd and the amount of KOAc may be selected as disclosed herein.

The inlet catalyst, outlet catalyst, and/or any additional catalyst refractory support may have a cross-sectional shape including, but not limited to, cylindrical, tubular, polylobular, ring, star, a trilobe, a quadrilobe, a cloverleaf shape, saddle, fluted, ridged, a multi-pointed star, a fluted ring, a hallow cylinder, a cogwheel, a spoked wheel, a multi-hole pellet, or a monolith, a T-fin shape, a Raschig ring shape, a spherical shape, or other shapes. In some aspects, the shape is spherical. The size of the spherical shape may range from 5 to 7 mm, on average. In some aspects, each of the inlet catalyst and outlet catalyst have a different size and a different layer thickness. For example, when the size of the spherical support is 5 mm, the layer is thicker than on a 6 mm or 7 mm shaped support.

Activity and selectivity of the catalysts are measured over a time of up to 200 hours. The catalysts are tested in a flow tube whose temperature is controlled by means of oil (reactor length 1200 mm, internal diameter 19 mm) at an absolute pressure of 9.8 bar and a space velocity (GHSV) of 4000-8000 standard m³/(m³*h) using the following gas composition: 60% by volume of ethylene, 19.5% by volume of argon, 13% by volume of acetic acid and 7.5% by volume of oxygen. The catalyst systems are tested in the temperature range from 130 to 180° C. (gas entry temperature upstream of the catalyst bed). To characterize the course of the reaction, the temperature profile is measured by means of a multi-point temperature sensor in the catalyst bed. The reaction products and unreacted starting materials are analyzed at the output of the reactor by means of on-line gas chromatography. The space-time yield of the catalyst system in gram of vinyl acetate monomer per hour and liter of catalyst (g (VAM)/l of cat.*h) is determined as a measure of the catalyst activity. The selectivity is determined via the ratio of vinyl acetate formed to ethylene reacted.

In addition to determination of the reaction products in the gas phase, the liquid reaction products are condensed in a vessel maintained at from 10 to 15° C. and the condensate obtained is analyzed by means of gas chromatography.

The acetoxylation of olefins, e.g., ethylene, may achieve favorable conversion of ethylene and favorable selectivity and productivity to vinyl acetate monomer. For purposes of the present invention, the term “conversion” refers to the amount of ethylene in the feed that is converted to a compound other than ethylene. Conversion is expressed as a percentage based on ethylene in the feed. The conversion may be at least 30%, e.g., at least 40%, or at least 60%. Although catalysts that have high conversions are desirable, such as at least 60%, in some embodiments a low conversion may be acceptable at high selectivity for VAM. It is, of course, well understood that in many cases, it is possible to compensate for conversion by appropriate recycle streams or use of larger reactors/more reactor tubes, but it is more difficult to compensate for poor selectivity.

Selectivity is expressed as a mole percent based on converted ethylene. It should be understood that each compound converted from ethylene has an independent selectivity and that selectivity is independent from conversion. For example, if 60 mole % of the converted ethylene is converted to vinyl acetate monomer, we refer to the vinyl acetate monomer selectivity as 60%. In one embodiment, catalyst selectivity to vinyl acetate monomer is at least 60%, e.g., at least 70%, at least 80%, or at least 90%. Preferably, the selectivity to vinyl acetate monomer is at least 80%, e.g., at least 85% or at least 88%. Preferred embodiments of the process also have low selectivity to undesirable products, such as heavy ends. The selectivity to these undesirable products preferably is less than 10%, e.g., less than 5% or less than 2%.

The selectivity and conversion relating to the reaction are functions of several variables including reactor temperature, component concentration, and the condition of the catalyst.

Deactivation of the catalyst, which routinely occurs over time due to buildup of tars and polymeric materials on the catalyst surface and/or to structural changes of the catalyst metals, may adversely affect the reaction process particularly with regard to selectivity. These changes in reactor performance may ultimately lead to compositional changes in the liquid stream entering the purification section of a vinyl acetate plant.

Generally, the performance of the zone loading may be reviewed by at least one of three measures: oxygen conversion, space-time yield, and the temperature through the reactor. In some aspects, two of these measures are used.

Oxygen conversion refers to the amount of oxygen provided to the reaction tube that is converted, similar to the measure of ethylene conversion. In some aspects, the oxygen conversion ranges from 35 to 55%, e.g., from 40 to 55%, or from 45 to 50%.

Space-time yield refers to the amount of product made per packed volume bed per unit time. In some aspects, the space-time yield (STY) is at least 800 g VAM/L/hour and may range from 800-1600 g VAM/L/hour, e.g., from 900 to 1500 g VAM/L/bour, or from 1000 to 1500 g VAM/L/hour.

The temperature across the reaction tube may range from 145 to 190° C., e.g. from 150 to 180° C., from 155° C. to 175° C., or from 160° C. to 170° C. Without being bound by theory, it is believed that the temperature may vary based on the catalyst age, with a newer catalyst typically tracking with a lower temperature than an older catalyst. A lower temperature across the reaction tube may indicate slower deactivation of the catalyst as compared to a higher temperature. A lower temperature may also indicate lower carbon dioxide selectivity which corresponds to more product made. Since ethylene is a relatively expensive component, operating at a lower temperature may burn less ethylene and may improve process efficiency. The temperature may be balanced with conversion, selectivity, and costs in replacing catalysts.

In some aspects, using the zone catalyst loading disclosed herein may advantageously achieve a desirable pressure drop across the reaction tube. In some aspects, the pressure drop may range from 12 to 30 psi, e.g., from 15 to 25 psi, or from 17.5 to 22.5 psi. The pressure drop may be measured by measuring the absolute pressure at the inlet of the reactor and the outlet of the reactor and then calculate the difference.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included.

As used herein and in the appended claims, the singular forms “a”, “an”, and “the” include plural referents unless the context clearly dictates otherwise. Thus, for example, reference to “a process” includes a plurality of such processes, and so forth.

Also, the words “comprise,” “comprising,” “include,” “including,” and “includes” when used in this specification and in the following claims are intended to specify the presence of stated features, integers, components, or steps, but they do not preclude the presence or addition of one or more other features, integers, components, steps, acts, or groups.

### Embodiments

Embodiment 1: A process for the acetoxylation of an olefin in a gaseous reaction stream containing an olefin, acetic acid, and an oxygen-containing gas, the process comprising: passing a reaction gas over at least two fixed catalyst zones, arranged in series, to form an acetoxylated olefin, wherein the catalyst zones are located in one or more reaction tubes arranged in parallel; wherein the at least two fixed catalyst zones comprise an inlet catalyst zone comprising an inlet catalyst and an outlet catalyst zone comprising an outlet catalyst; wherein the inlet catalyst zone comprises from 5 to 70% of the catalyst loading and the outlet catalyst zone comprises from 30 to 95% of the catalyst loading, based on 100% by weight of catalyst loading of all catalyst zones; and wherein at least one of the following conditions is met:(a) the inlet catalyst comprises greater than 7 g/L:Pd; (b) the outlet catalyst comprises greater than 8.3 g/L Pd; and/or (c) the outlet catalyst comprises from 7.5 to 8.1 g/L Pd.
Embodiment 2: The process of Embodiment 1, wherein the inlet catalyst zone comprises from 40 to 70% by weight of the catalyst loading.
Embodiment 3: The process of Embodiment 1 or 2, wherein condition (b) and/or (c) is met and wherein the inlet catalyst comprises from 4 to 15 g/L Pd and from 2 to 8 g/L Au.
Embodiment 4: The process of any of the preceding Embodiments, wherein the outlet catalyst zone comprises from 30 to 50% by weight of the catalyst loading.
Embodiment 5: The process of any of the preceding Embodiments, wherein condition (a) is met and wherein the outlet catalyst comprises from 4 to 15 g/L Pd and from 2 to 8 g/L Au.
Embodiment 6: The process of Embodiment 1, wherein the inlet catalyst zone comprises from 5 to 40% by weight of the catalyst loading.
Embodiment 7: The process of Embodiment 6, wherein the inlet catalyst has a weight ratio of Au to Pd from 0.3:1 to 1.5:1.
Embodiment 8: The process of Embodiment 6 or 7, wherein the inlet catalyst has a layer thickness from 100 to 300 micrometers.
Embodiment 9: The process of any of Embodiments 6-8, wherein the inlet catalyst zone contains at least two catalysts in series and wherein the weight ratio of the two catalysts is from 1:5 to 5:1.
Embodiment 10: The process of any of Embodiments 6-9, wherein the outlet catalyst zone comprises from 60 to 95% by weight of the catalyst loading.
Embodiment 11: The process of any of Embodiments 6-10, wherein the outlet catalyst has a weight ratio of Au to Pd of 0.3:1 to 1.5:1.
Embodiment 12: The process of any of the preceding Embodiments, wherein the inlet catalyst has a different metal loading than the outlet catalyst.
Embodiment 13: The process of any of the preceding Embodiments, wherein the inlet catalyst zone comprises a catalyst having a different size than the outlet catalyst zone.
Embodiment 14: The process of any of the preceding Embodiments, wherein the inlet catalyst and outlet catalyst each comprise less than 2 wt. % Pd.
Embodiment 15: The process of any of the preceding Embodiments, wherein the inlet catalyst and outlet catalyst each comprise from 25 to 55 g/L potassium acetate.
Embodiment 16: The process of any of the preceding Embodiments, wherein the olefin is ethylene and wherein the selectivity to vinyl acetate monomer is at least 60%.
Embodiment 17: The process of any of the preceding Embodiments, wherein the olefin is ethylene and wherein the conversion of ethylene is at least 60%.
Embodiment 18: The process of any of the preceding Embodiments, wherein the olefin is ethylene and the selectivity to heavy ends is less than 10%.
Embodiment 19: The process of any of the preceding Embodiments, wherein the inlet catalyst and the outlet catalyst have a spherical shape.
Embodiment 20: The process of any of the preceding Embodiments, wherein the olefin is ethylene and the oxygen conversion is from 35 to 55%.
Embodiment 21: The process of Embodiment 1, wherein only condition a) is met.
Embodiment 22: The process of Embodiment 1, wherein only condition b) is met.
Embodiment 23: The process of Embodiment 1, wherein only condition c) is met.
Embodiment 24: The process of Embodiment 1, wherein conditions a) and b) are met.
Embodiment 25: The process of Embodiment 1, wherein conditions a) and c) are met.

While the invention has been described in detail, modifications within the spirit and scope of the invention will be readily apparent to those of skill in the art. It should be understood that aspects of the invention and portions of various embodiments and various features recited above and/or in the appended claims may be combined or interchanged either in whole or in part.

In the foregoing descriptions of the various embodiments, those embodiments which refer to another embodiment may be appropriately combined with other embodiments as will be appreciated by one of ordinary skill in the art. Furthermore, those of ordinary skill in the art will appreciate that the foregoing description is by way of example only, and is not intended to limit the invention. All US patents and publications cited herein are incorporated by reference in their entirety.

1. A process for the acetoxylation of an olefin in a gaseous reaction stream containing an olefin, acetic acid, and an oxygen-containing gas, the process comprising:
passing a reaction gas over at least two fixed catalyst zones, arranged in series, to form an acetoxylated olefin,
wherein the catalyst zones are located in one or more reaction tubes arranged in parallel;
wherein the at least two fixed catalyst zones comprise an inlet catalyst zone comprising an inlet catalyst and an outlet catalyst zone comprising an outlet catalyst;
wherein the inlet catalyst zone comprises from 5 to 70% of the catalyst loading and the outlet catalyst zone comprises from 30 to 95% of the catalyst loading, based on 100% by weight of catalyst loading of all catalyst zones; and wherein at least one of the following conditions is met:
(a) the inlet catalyst comprises greater than 7 g/L Pd;
(b) the outlet catalyst comprises greater than 8.3 g/L Pd; and/or
(c) the outlet catalyst comprises from 7.5 to 8.1 g/L Pd.

2. The process of claim 1, wherein the inlet catalyst zone comprises from 40 to 70% by weight of the catalyst loading.

3. The process of claim 1, wherein condition (b) and/or (c) is met and wherein the inlet catalyst comprises from 4 to 15 g/L Pd and from 2 to 8 g/L Au.

4. The process of f claim 1, wherein the outlet catalyst zone comprises from 30 to 50% by weight of the catalyst loading.

5. The process of claim 1, wherein condition (a) is met and wherein the outlet catalyst comprises from 4 to 15 g/L Pd and from 2 to 8 g/L Au.

6. The process of claim 1, wherein the inlet catalyst zone comprises from 5 to 40% by weight of the catalyst loading.

7. The process of claim 6, wherein the inlet catalyst has a weight ratio of Au to Pd from 0.3:1 to 1.5:1.

8. The process of claim 6, wherein the inlet catalyst has a layer thickness from 100 to 300 micrometers.

9. The process of claim 6, wherein the inlet catalyst zone contains at least two catalysts in series and wherein the weight ratio of the two catalysts is from 1:5 to 5:1.

10. The process of claim 6, wherein the outlet catalyst zone comprises from 60 to 95% by weight of the catalyst loading.

11. The process of claim 6, wherein the outlet catalyst has a weight ratio of Au to Pd of 0.3:1 to 1.5:1.

12. The process of claim 1, wherein the inlet catalyst has a different metal loading than the outlet catalyst.

13. The process of am claim 1, wherein the inlet catalyst zone comprises a catalyst having a different size than the outlet catalyst zone.

14. The process of claim 1, wherein the inlet catalyst and outlet catalyst each comprise less than 2 wt. % Pd.

15. The process of claim 1, wherein the inlet catalyst and outlet catalyst each comprise from 25 to 55 g/L potassium acetate.

16. The process of claim 1, wherein at least one the following is met:
a) the olefin is ethylene and wherein the selectivity to vinyl acetate monomer is at least 60%;
b) wherein the olefin is ethylene and wherein the conversion of ethylene is at least 60%;
c) wherein the olefin is ethylene and the selectivity to heavy ends is less than 10%; and/or
d) wherein the olefin is ethylene and the oxygen conversion is from 35 to 55%.

17-18. (canceled)

19. The process of claim 1, wherein the inlet catalyst and the outlet catalyst have a spherical shape.

20. (canceled)

21. The process of claim 1, wherein only one condition is met.

22-23. (canceled)

24. The process of claim 1, wherein conditions a) and b) are met.

25. The process of claim 1, wherein conditions a) and c) are met.
